(19) United States
(12) Patent Application Publication
(10) Pub. No.: US 2019/0077893 A1
(43) Pub. Date: Mar. 14, 2019

(54) CROSS-COPOLYMER AND MEDICAL SINGLE-LAYER TUBE INCLUDING SAME

(71) Applicant: Denka Company Limited, Chuo-ku, Tokyo (JP)

(72) Inventors: Yushi KUMAGAI, Ichihara-shi, Chiba (JP); Masaru HASEGAWA, Ichihara-shi, Chiba (JP); Tetsuo NOGUCHI, Ichihara-shi, Chiba (JP)

(73) Assignee: Denka Company Limited, Chuo-ku, Tokyo (JP)

(21) Appl. No.: 16/064,703

(22) PCT Filed: Oct. 25, 2016
(86) PCT No.: PCT/JP2016/081525

§ 371 Date: Nov. 26, 2018

(30) Foreign Application Priority Data
Dec. 25, 2015 (JP) 2015-254652

(51) Int. Cl. C08F 212/08; C08F 212/34; C08F 210/02; A61L 31/04

(52) U.S. Cl. CPC C08F 212/08; C08F 212/34; C08F 2800/20; A61L 31/048; C08F 2800/10; C08F 210/02

(57) ABSTRACT

[Problem] To provide a cross-polymer having excellent softness, tensile properties, transparency and anti-blocking properties, and a medical single-layer tube using the same. [Solution] A cross-copolymer containing, by 75-95 mass %, main chains comprising aromatic vinyl/olefin-based copolymers comprising 8.99-15.99 mol % of aromatic vinyl monomer units, 84-91 mol % of olefin monomer units and 0.01-0.5 mol % of aromatic polyene monomer units; and by 5-25 mass %, cross-chains comprising polymers comprising aromatic vinyl monomer units; wherein, based on differential scanning calorimetry (DSC) in which the cross-copolymer is cooled to −50° C. in a 30 mL/min nitrogen flow, then heated to 180° C. at a temperature increase rate of 10° C./min, re-cooled to −50° C., and heated to 180° C. at a temperature increase rate of 10° C./min, the cross-copolymer has a top temperature of melting peak of 60-80° C., and a heat of fusion of 45-75 J/g, as calculated from the area of the DSC curve between −20° C. and 130° C. by using a straight line drawn between points on the DSC curve at −20° C. and 130° C.

### TECHNICAL FIELD

The present invention relates to a cross-copolymer having excellent softness, tensile properties, transparency, and anti-blocking properties, and a medical single-layer tube using the same.

### BACKGROUND ART

In recent years, thermoplastic elastomers, which have excellent productivity, have come to be widely used in applications, such as automotive components, household appliance components, medical components, and sundry goods, which have conventionally been primarily formed of vulcanized rubber. Among these thermoplastic elastomers, many novel thermoplastic elastomers having properties that are sought in various applications have been proposed. For example, Patent Document 1 proposes a so-called cross-copolymer that is obtained by copolymerizing a small amount of divinvylbenzene with a styrene-ethylene copolymer and introducing polystyrene (cross-chain) via a vinyl group in the divinylbenzene unit. The cross-copolymers obtained by this method are branched block copolymers having styrene-ethylene copolymer chains as soft segments and polystyrenes as hard segments, and are materials having very excellent scratch resistance and moldability.

Medical tubes, which are medical components, require various properties such as softness, transparency, and bend resistance (kink resistance), as well as drug amount standardization properties such that there is little drug adsorption or absorption and drugs can be transported in fixed quantities, and squeeze resistance (shape recovery properties, wear resistance etc.), which makes the medical tubes suitable for use in infusion pump circuits, and further thereto, excellent radiation resistance against gamma rays and electron rays used for sterilization. In response to these requirements, Patent Document 2 proposes a medical tube wherein, after the tube is molded, the surface is crosslinked by irradiation with electron rays, thereby resulting in a medical tube having softness, transparency, low drug adsorption and absorption, pump circuit suitability, and chemical stability, as well as having excellent kink resistance, suppressing blocking and having sufficient heat resistance to withstand various sterilization methods. Patent Document 3 proposes a multilayer tube in which a support layer that occupies 50% or more of the tube thickness is formed from a cross-copolymer having sufficient softness, and an inner layer is formed from a material having low blocking properties, thereby improving the occlusion due to tight contact between the inner walls when the tube is bent or clamped with forceps or the like. In recent years, there has been a movement to make medical components disposable, and they are often incinerated after use in order to prevent biohazards, so it has become important to use non-flexible vinyl chloride materials that do not generate chlorine compounds as gases when incinerating.
Patent Document 1: JP 2009-102515 A
Patent Document 2: JP 2013-202133 A
Patent Document 3: WO 2013/137326 A1

### SUMMARY OF THE INVENTION

Under these circumstances, if the anti-blocking properties could be further improved while maintaining the excellent properties of cross-copolymers such as softness, tensile properties, and transparency, then they could be used in single layers, particularly when used as medical tubes, and their value would also increase for other applications. Thus, further improvements have been sought.

The present invention provides a cross-polymer having excellent softness, tensile properties, transparency, and anti-blocking properties, and a medical single-layer tube using the same.

The gist of the present invention is as follows.
(1) A cross-copolymer containing, by 75-95 mass %, main chains comprising aromatic vinyl/olefin-based copolymers comprising 8.99-15.99 mol % of aromatic vinyl monomer units, 84-91 mol % of olefin monomer units and 0.01-0.5 mol % of aromatic polyene monomer units; and by 5-25 mass %, cross-chains comprising polymers comprising aromatic vinyl monomer units; wherein, based on differential scanning calorimetry (DSC) in which the cross-copolymer is cooled to −50° C. in a 30 mL/min nitrogen flow, then heated to 180° C. at a temperature increase rate of 10° C./min, re-cooled to −50° C., and heated to 180° C. at a temperature increase rate of 10° C./min, the cross-copolymer has Tm (a top temperature of melting peak) of 60-80° C., and a heat of fusion of 45-75 J/g, as calculated from the area of the DSC curve between −20° C. and 130° C. by using a straight line drawn between points on the DSC curve at −20° C. and 130° C.
(2) In one embodiment, the present invention may be the cross-copolymer as in (1), wherein the composition distribution of the aromatic vinyl/olefin-based copolymers forming the main chains is such that aromatic vinyl/olefin-based copolymers comprising at least 85 mol % and at most 92 mol % of olefin monomer units are contained in an amount of at least 50 mass %, aromatic vinyl/olefin-based copolymers comprising less than 85 mol % of olefin monomer units are contained in an amount of less than 35 mass %, and aromatic vinyl/olefin-based copolymers comprising more than 92 mol % of olefin monomer units are contained in an amount of less than 15 mass %.
(3) A cross-copolymer as in (1) or (2), wherein Tm (the top temperature of melting peak) is 65-73° C. and the heat of fusion is 50-70 J/g is preferable.
(4) A cross-copolymer as in any one of (1) to (3), wherein the aromatic vinyl monomer units are styrene is preferable.
(5) A cross-copolymer as in any one of (1) to (4), wherein the olefin monomer units are ethylene is preferable.
(6) Additionally, the present invention is a medical single-layer tube containing the cross-copolymer as in any one of (1) to (5).

According to the present invention, it is possible to provide a cross-polymer having excellent softness, tensile properties, transparency, and anti-blocking properties, and a medical single-layer tube using the same.

### MODES FOR CARRYING OUT THE INVENTION

Herebelow, embodiments of the present invention will be explained in detail. In the present specification and the claims, the recitation “A-B” refers to the range of values that are at least A and at most B.

### [Cross-Copolymer]

The cross-copolymer has a structure including main chains comprising aromatic vinyl/olefin-based copolymers comprising aromatic vinyl monomer units, olefin monomer units, and aromatic polyene monomer units, and cross-chains comprising polymers comprising aromatic vinyl monomer units, wherein the polymers comprising aromatic vinyl monomer units are bound via aromatic polyene monomer units in the main chains.

### (Main Chains)

Examples of aromatic vinyl monomer units include units derived from styrene-based monomers such as styrene and various substituted styrenes, e.g., p-methylstyrene, m-methylstyrene, o-methylstyrene, o-t-butylstyrene, m-t-butylstyrene, p-t-butylstyrene, p-chlorostyrene, and o-chlorostyrene. Among these, styrene units, p-methylstyrene units, and p-chlorostyrene units are preferred, and styrene units are particularly preferred. These aromatic vinyl monomer units may be of a single type, or may be a combination of two or more types.

Examples of olefin monomer units include units derived from α-olefin monomers and cyclic olefin-based monomers, such as ethylene and α-olefins having 3 to 20 carbon atoms, e.g., propylene, 1-butene, 1-hexene, 4-methyl-1-pentene, 1-octene, vinylcyclohexane, and cyclic olefins, i.e. cyclopentene, norbornene, etc. Preferably, a mixture of ethylene units, propylene units, 1-butene units, 1-hexene units and 1-octene units is used, and ethylene units are particularly preferred.

Examples of aromatic polyene monomer units include units derived from aromatic polyene monomers which are aromatic polyenes having at least 10 and at most 30 carbon atoms, a plurality of double bonds (vinyl groups) and a single or a plurality of aromatic groups, e.g., o-divinylbenzene, p-divinylbenzene, m-divinylbenzene, 1,4-divinylnaphthalene, 3,4-divinylnaphthalene, 2,6-divinylnaphthalene, 1,2-divinyl-3,4-dimethylbenzene, 1,3-divinyl-4,5,8-tributylnaphthalene etc. Preferably, one or a mixture of two or more of ortho-divinylbenzene units, para-diviylbenzene units and meta-divinylbenzene units are used.

The proportional amounts of the structural units contained in the aromatic vinyl/olefin-based copolymers are 8.99-15.99 mol % for the aromatic vinyl monomer units, 84-91 mol % for the olefin monomer units and 0.01-0.5 mol % for the aromatic polyene monomer units, preferably 9.97-13.97 mol % for the aromatic vinyl monomer units, 86-90 mol % for the olefin monomer units and 0.03-0.3 mol % for the aromatic polyene monomer units

When the aromatic vinyl monomer units are contained in an amount of 8.99 mol % or more, the crystalline structure derived from the olefin chain structures is suppressed, thereby increasing the softness and the transparency. The aromatic vinyl monomer units are preferably contained in an amount of 9.97 mol % or more. When the aromatic vinyl monomer units are contained in an amount of 15.99 mol % or less, the tensile properties and the anti-blocking properties are improved by the crystalline structure derived from the olefin chain structure. The aromatic vinyl monomer units are preferably contained in an amount of 13.97 mol % or less.

When the olefin monomer units are contained in an amount of 84 mol % or more, the tensile properties and the anti-blocking properties are improved by the olefin chain structures. The olefin monomer units are preferably contained in an amount of 86 mol % or more. Additionally, when the olefin monomer units are contained in an amount of 91 mol % or less, the crystalline structure derived from the olefin chain structures is suppressed, thereby increasing the softness and the transparency of the cross-copolymer. The olefin monomer units are preferably contained in an amount of 90 mol % or less.

When the aromatic polyene monomer units are contained in an amount of 0.01 mol % or more, polymer cross-chains comprising the aromatic vinyl monomer units can be formed, thereby improving the tensile properties. The aromatic polyene monomer units are preferably contained in an amount of 0.03 mol % or more. When the aromatic polyene monomer units are contained in an amount of 0.5 mol % or less, it is possible to suppress molecular weight increases due to crosslinking reactions, thereby resulting in good production stability and moldability. The aromatic polyene monomer units are preferably contained in an amount of 0.3 mol % or less.

As preferable ranges for the copolymer composition distribution of the aromatic vinyl/olefin-based copolymers is such that, for example, aromatic vinyl/olefin-based copolymers comprising less than 85 mol % of olefin monomer units are contained in an amount of less than 35 mass %, aromatic vinyl/olefin-based copolymers comprising 85-92 mol % of olefin monomer units are contained in an amount of 50 mass % or more, and aromatic vinyl/olefin-based copolymers comprising more than 92 mol % of olefin monomer units are contained in an amount of less than 15 mass %.

While the weight-average molecular weight of the aromatic vinyl/olefin-based copolymers is not particularly limited, it should preferably be 30,000 to 300,000, more preferably 50,000 to 200,000 in view of the moldability. In the present specification, the weight-average molecular weight refers to polystyrene-converted values measured by gel permeation chromatography (GPC), the measurements being made under the below-indicated measurement conditions.
Equipment: HLC-8220 (manufactured by Toso)
Column: Shodex GPC KF-404HQ, four in a serial arrangement
Temperature: 40° C.
Detection: Differential refractive index
Solvent: Tetrahydrofuran
Calibration curve: Produced using standard polystyrene (PS)

### (Cross-Chains)

The polymers comprising aromatic vinyl monomer units forming the cross-chains may be polymers comprising aromatic vinyl monomer units of a single type, or may be copolymers comprising aromatic vinyl monomer units of two or more types. The aromatic vinyl monomer units may be of the same type as those in the main chain mentioned above.

While the weight-average molecular weights of the polymers comprising aromatic vinyl monomer units forming the cross-chains are not particularly limited, they should preferably be 3,000 to 150,000, more preferably 5,000 to 70,000 in view of the moldability.

### (Cross-Copolymer)

The cross-copolymer is a copolymer comprising, by 75-95 mass %, main chains comprising aromatic vinyl/olefin-based copolymers, and by 5-25 mass %, cross-chains comprising polymers comprising aromatic vinyl monomer units. When the main chains comprising the aromatic vinyl/olefin-based copolymers are present in an amount of 75 mass % or more, the softness is improved. The main chains comprising aromatic vinyl/olefin-based copolymers are preferably present in an amount of 80 mass % or more. When the main chains comprising the aromatic vinyl/olefin-based copolymers are present in an amount of 95 mass % or less, the tensile properties and the anti-blocking properties are improved. The main chains comprising aromatic vinyl/olefin-based copolymers are preferably present in an amount of 90 mass % or less. When the cross-chains are present in an amount of 5 mass % or more, the tensile properties and the anti-blocking properties are improved. The cross-chains are preferably present in an amount of 10 mass % or more. When the cross-chains are present in an amount of 25 mass % or less, the softness and the transparency are improved. The cross-chains are preferably present in an amount of 20 mass % or less.

Based on differential scanning calorimetry (DSC) in which the cross-copolymer is cooled to −50° C. in a 30 mL/min nitrogen flow, then heated to 180° C. at a temperature increase rate of 10° C./min, re-cooled to −50° C. and heated to 180° C. at a temperature increase rate of 10° C./min, Tm (the top temperature of the melting peak) (hereinafter also referred to simply as “Tm (the melting peak temperature)”) is 60° C. or higher and 80° C. or lower, preferably 65° C. or higher and 73° C. or lower. When Tm (the melting peak temperature) is 60° C. or higher, the tensile properties and the anti-blocking properties are improved by the crystalline structure derived from the olefin chain structures. Tm (the melting peak temperature) is preferably 65° C. or higher. Additionally, when Tm (the melting peak temperature) is 80° C. or lower, the crystalline structure derived from the olefin chain structures is suppressed, thereby increasing the softness and the transparency. Tm (the melting peak temperature) is preferably 73° C. or lower.

Further, Tm (the melting peak temperature) refers to the melting point of the crystalline structure derived from the olefin chain structures in the aromatic vinyl/olefin-based copolymers.

Based on differential scanning calorimetry (DSC) in which the cross-copolymer is cooled to −50° C. in a 30 mL/min nitrogen flow, then heated to 180° C. at a temperature increase rate of 10° C./min, re-cooled to −50° C., and heated to 180° C. at a temperature increase rate of 10° C./min, the heat of fusion, as calculated from the area of the DSC curve between −20° C. and 130° C. using a straight line drawn between points on the DSC curve at −20° C. and 130° C. (hereinafter also referred to simply as “heat of fusion”), is 45-75 J/g, preferably 50-70 J/g. When the heat of fusion is 45 J/g or higher, the tensile properties and the anti-blocking properties are improved by the crystalline structure derived from the olefin chain structures. The heat of fusion is preferably 50 J/g or higher. When the heat of fusion is 75 J/g or lower, the crystalline structure derived from the olefin chain structures is suppressed, thereby increasing the softness and the transparency. The heat of fusion is preferably 70 J/g or lower.

Further, the heat of fusion refers to the heat of fusion of the crystalline structure derived from the olefin chain structures in the aromatic vinyl/olefin-based copolymers, and is observed between −20° C. and 130° C.

The differential scanning calorimetry (DSC) was performed by measuring 6 mg of the cross-copolymer using a Seiko Instruments DSC6200. The aromatic vinyl/olefin-based copolymers have a crystalline structure derived from the aromatic vinyl/olefin structures in addition to the crystalline structure derived from the olefin chain structures. Since the crystallization rate of the crystalline structure derived from the aromatic vinyl/olefin structures is slow, it is possible to observe just the crystalline structure derived from the olefin chain structures by cooling the cross-copolymer to −50° C. in a 30 mL/min nitrogen flow, then heating to 180° C. at a temperature increase rate of 10° C./min, re-cooling to −50° C., and heating to 180° C. at a temperature increase rate of 10° C./min.

### [Production Method]

The cross-copolymer production method according to the present embodiment will be explained. There are no particular limitations regarding the polymerization format, and known methods such as solution polymerization and bulk polymerization may be used, but solution polymerization is more preferred due to the higher level of freedom in controlling the polymerization to obtain a desired cross-copolymer.

The polymerization method is not particularly limited as long as the desired cross-copolymer can be obtained, but the cross-copolymer may be produced by means of a production method involving a two-stage polymerization process comprising a coordination polymerization step in which a coordination polymerization catalyst is used to polymerize the aromatic vinyl/olefin-based copolymers, and an anionic polymerization step in which an anionic polymerization initiator is used to induce polymerization of the aromatic vinyl/olefin-based copolymers obtained in the coordination polymerization step in the co-presence of aromatic vinyl monomers, thereby resulting in the cross-copolymer, which has a structure with polymers comprising aromatic vinyl monomer units, as cross-chains, on vinyl groups remaining on the aromatic polyene monomer units of main chains.

### (Coordination Polymerization Step)

The coordination polymerization step will be explained in detail. As the coordination polymerization catalyst, it is possible to use a single-site coordination polymerization catalyst composed of a transition metal compound and a promoter. Methylaluminoxane may be favorably used as the promoter for promoting the activity of the single-site coordination catalyst. Additionally, alkylaluminum may be favorably used for removing water contained in the solvent or in the monomer raw materials and preventing the single-site coordination polymerization catalyst from being poisoned by reacting with the water and thereby reducing the catalytic function. Since the presence of a polar functional group will poison the single-site coordination polymerization catalyst, the solvent that is used is preferably a hydrocarbon-based solvent such as cyclohexane, methylcyclohexane, toluene or ethylbenzene, or an aromatic hydrocarbon-based solvent. The amount of the solvent that is added is preferably 200-900 parts by mass with respect to 100 parts by mass of the resulting copolymer. Two hundred parts by mass or more is preferable for controlling the polymerization solution viscosity and the reaction rate, and 900 parts by mass or less is favorable in terms of the productivity.

The steps in the coordination polymerization are not particularly limited, but the polymerization must be performed while controlling the copolymer composition distribution of the aromatic vinyl/olefin-based copolymers to be within a specific range so that the cross-copolymer will have the above-mentioned Tm (the melting peak temperature) (60-80° C.) and heat of fusion (45-75 J/g) for the DSC curve. In other words, it is preferable to use a method in which the copolymer composition distribution is controlled to be within a specific range by appropriately adjusting the rate of addition of olefin monomers in accordance with the polymerization rate, or by adding more of the aromatic vinyl monomers as needed or adding it in batches, while taking into consideration the reactivity ratio between the aromatic vinyl monomers and the polyolefin monomers. It is preferable to control the polymerization rate while appropriately adjusting the polymerization temperature, the stirring conditions, the pressure conditions or the like, since this allows the copolymer composition distribution to be more precisely controlled.

### (Anionic Polymerization Step)

The anionic polymerization step will be explained in detail. In the anionic polymerization step, an anionic polymerization initiator is used to induce polymerization of the aromatic vinyl/olefin-based copolymers obtained in the coordination polymerization step in the co-presence of aromatic vinyl monomers, thereby synthesizing a cross-copolymer having a structure with polymers comprising aromatic vinyl monomer units, as cross-chains, on vinyl groups remaining on the aromatic polyene monomer units of main chains. The aromatic vinyl/olefin-based copolymers obtained in the coordination polymerization step may be separated from the polymerization solution after the coordination polymerization and purified for use in the anionic polymerization step using any method, such as a method of precipitation in a poor solvent such as methanol, a method of precipitation by evaporation of a solvent by a heated roller or the like (drum dryer method), a method of concentrating the solution with a concentrator followed by removal of the solvent by a vent-type extruder, a method of dispersing the solution in water and thermally removing the solvent by blowing in steam to recover the copolymer (steam stripping method), or a clamshell forming method. Alternatively, the polymerization solution containing the aromatic vinyl/olefin-based copolymers may be used in the anionic polymerization step without separating the aromatic vinyl/olefin-based copolymers from the polymerization solution and purifying them, and such a method is favorable in terms of productivity. As the anionic polymerization initiator, it is possible to use a known anionic polymerization initiator such as n-butyllithium or sec-butyllithium. As the aromatic vinyl monomers, it is possible to use the aromatic vinyl monomers remaining in the polymerization solution after coordination polymerization, directly as they are. Additionally, it is possible to obtain the target cross-copolymer by adding the necessary amount before beginning anionic polymerization, or by adding more as needed or adding it in batches during the anionic polymerization.

### (Recovery Step)

The method for recovering the cross-copolymer is not particularly limited, and it is possible to use a known method such as a method of precipitation in a poor solvent such as methanol, a method of precipitation by evaporation of a solvent by a heated roller or the like (drum dryer method), a method of dispersing the solution in water and thermally removing the solvent by blowing in steam to recover the copolymer (steam stripping method), or a clamshell forming method. Alternatively, there is a method of using a gear pump to continuously feed the polymerization solution into a twin-screw devolatilizing extruder to remove the polymerization solution. This method is economically preferable, since it allows the polymerization solvent to be reused by condensing and recovering the removed components, including the polymerization solvent, in a condenser or the like, and purifying the condensate in a distillation column.

[Medical Single-Layer Tube]

The medical single-layer tube includes the above-described cross-copolymer. The production method thereof is not particularly limited, and it is possible to use a known method such as extrusion molding, injection molding, blow molding, or rotational molding.

### EXAMPLES

Hereinbelow, the present invention will be explained by providing examples and comparative examples, but these are merely exemplary and should not be construed as limiting the subject matter of the present invention.

### [Synthesis of Cross-Copolymer]

In the following Synthesis Examples 1-7, rac-dimethylmethylene bis(4,5-benzo-1-indenyl)zirconium dichloride (Chemical Formula 1) was used as the coordination polymerization catalyst.

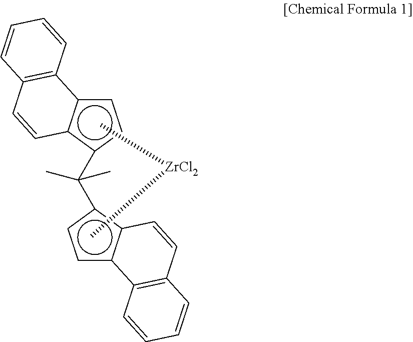

### Synthesis Example 1

### Synthesis of Cross-Copolymer (I)

### (Coordination Polymerization Step)

Polymerization was performed using an autoclave with a capacity of 50 L, equipped with a stirrer and a heating/cooling jacket. 20.0 kg of cyclohexane, 2.43 kg of styrene, and divinylbenzene manufactured by Nippon Steel Chemical (meta- and para-mixture, 84 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was fed until the pressure reached 0.665 MPaG. Thereafter, 50 mL of a toluene solution into which were dissolved 80 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.665 MPaG. When the consumed amount of ethylene reached 1.00 kg, a small amount of the polymerization solution was sampled. When the consumed amount of ethylene reached 2.00 kg, a small amount of the polymerization solution was sampled.

When the consumed amount of ethylene reached 2.10 kg, the supplementation of the ethylene was temporarily stopped, and after the ethylene was consumed until the pressure reached 0.565 MPaG, polymerization was performed while maintaining the pressure at 0.565 MPaG. When the consumed amount of ethylene reached 2.80 kg, the supplementation of the ethylene was temporarily stopped, and after the ethylene was consumed until the pressure reached 0.465 MPaG, polymerization was performed while maintaining the pressure at 0.465 MPaG. When the consumed amount of ethylene reached 3.00 kg, a small amount of the polymerization solution was sampled.

When the consumed amount of ethylene reached 3.30 kg, the supplementation of the ethylene was temporarily stopped, and after the ethylene was consumed until the pressure reached 0.415 MPaG, polymerization was performed while maintaining the pressure at 0.415 MPaG. When the consumed amount of ethylene reached 3.50 kg, the supplementation of the ethylene was temporarily stopped, and after the ethylene was consumed until the pressure reached 0.365 MPaG, polymerization was performed while maintaining the pressure at 0.365 MPaG. When the consumed amount of ethylene reached 3.70 kg, the supplementation of the ethylene was temporarily stopped, and after the ethylene was consumed until the pressure reached 0.265 MPaG, polymerization was performed while maintaining the pressure at 0.265 MPaG. When the consumed amount of ethylene reached 3.80 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization.

The sampled polymerization solution was mixed with large quantities of methanol to precipitate the resin components, which were filtered and dried to obtain samples of the aromatic vinyl/olefin-based copolymers, and the resin rates in the polymerization solution were determined. From the obtained samples, the respective monomer unit contents (mol %) in the aromatic vinyl/olefin-based copolymers were determined by analysis of the samples taken during the coordination polymerization. Additionally, the samples taken after stopping the coordination polymerization were analyzed to determine the respective monomer unit contents (mol %) and the weight-average molecular weight of the aromatic vinyl/olefin-based copolymers. The analysis results are shown in Tables 1 and 2.

The methods for measuring the “resin rate”, the “monomer unit content”, the “weight-average molecular weight”, the “main chain and cross-chain contents” and the like will be discussed below.

### (Anionic Polymerization Step)

After coordination polymerization, when the internal temperature fell to 70° C., 210 mmol of n-butyllithium (hexane solution) was fed from a catalyst tank into the polymerization tank together with nitrogen gas. Anionic polymerization began immediately and the internal temperature rose from 70° C. to 75° C. at one point. The internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (I) in pellet form. The resulting cross-copolymer (I) was analyzed to determine the amount (mass %) of the aromatic vinyl/olefin-based copolymers, forming the main chains, contained therein, and the amount (mass %) of polymers comprising aromatic vinyl monomer units, forming the cross-chains, contained therein. Additionally, due to the nature of anionic polymerization, the polymers comprising aromatic vinyl monomer units forming the cross-chains and the polymers comprising aromatic vinyl monomer units not bonding to the main chains have approximately the same molecular weight, so the weight-average molecular weight of the cross-chains was determined by separating the polymers comprising aromatic monomer units that are not bound to main chains, which are produced in small amounts as byproducts of the anionic polymerization step, and measuring the weight-average molecular weight thereof. The analysis results thereof are shown in Table 1. Additionally, the differential scanning calorimetry (DSC) results obtained by measurements using a Seiko Instruments DSC6200 are shown in Table 4. The methods for measuring Tm (the melting peak temperature) and the heat of fusion by differential scanning calorimetry (DSC) will be described below.

### Synthesis Example 2

### Synthesis of Cross-Copolymer (II)

(Coordination Polymerization Step)

Polymerization was performed using an autoclave with a capacity of 50 L, equipped with a stirrer and a heating/cooling jacket. 20.2 kg of cyclohexane, 2.48 kg of styrene, and divinylbenzene manufactured by Nippon Steel Chemical (meta- and para-mixture, 84 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. ethylene was introduced, and the pressure reached 0.665 MPaG. Thereafter, 50 mL of a toluene solution into which were dissolved 80 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.665 MPaG. When the consumed amount of ethylene reached 1.00 kg, a small amount of the polymerization solution was sampled, the supplementation of the ethylene was temporarily stopped, and after the ethylene was consumed until the pressure reached 0.565 MPaG, polymerization was performed while maintaining the pressure at 0.565 MPaG. When the consumed amount of ethylene reached 1.60 kg, the supplementation of the ethylene was temporarily stopped, and after the ethylene was consumed until the pressure reached 0.515 MPaG, polymerization was performed while maintaining the pressure at 0.515 MPaG.

When the consumed amount of ethylene reached 2.00 kg, a small amount of the polymerization solution was sampled.

The supplementation of the ethylene was temporarily stopped, and after the ethylene was consumed until the pressure reached 0.465 MPaG, polymerization was performed while maintaining the pressure at 0.465 MPaG. When the consumed amount of ethylene reached 2.30 kg, the supplementation of the ethylene was temporarily stopped, and after the ethylene was consumed until the pressure reached 0.415 MPaG, polymerization was performed while maintaining the pressure at 0.415 MPaG. When the consumed amount of ethylene reached 2.70 kg, the supplementation of the ethylene was temporarily stopped, and after the ethylene was consumed until the pressure reached 0.365 MPaG, polymerization was performed while maintaining the pressure at 0.365 MPaG. When the consumed amount of ethylene reached 3.00 kg, a small amount of the polymerization solution was sampled.

When the consumed amount of ethylene reached 3.10 kg, the supplementation of the ethylene was temporarily stopped, and after the ethylene was consumed until the pressure reached 0.315 MPaG, polymerization was performed while maintaining the pressure at 0.315 MPaG. When the consumed amount of ethylene reached 3.30 kg, the supplementation of the ethylene was temporarily stopped, and after the ethylene was consumed until the pressure reached 0.265 MPaG, polymerization was performed while maintaining the pressure at 0.265 MPaG. When the consumed amount of ethylene reached 3.40 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C. 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (II) in pellet form. The resulting cross-copolymer (II) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weight of the cross-chains. The analysis results are shown in Table 1. Additionally, the differential scanning calorimetry (DSC) results are shown in Table 4.

### Synthesis Example 3

### Synthesis of Cross-Copolymer (III)

### (Coordination Polymerization Step)

Polymerization was performed using an autoclave with a capacity of 50 L. equipped with a stirrer and a heating/cooling jacket. 20.0 kg of cyclohexane, 2.25 kg of styrene, and divinylbenzene manufactured by Nippon Steel Chemical (meta- and para-mixture, 84 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was introduced and the pressure reached 0.665 MPaG. Thereafter, 50 mL of a toluene solution into which were dissolved 80 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.665 MPaG. When the consumed amount of ethylene reached 1.00 kg, a small amount of the polymerization solution was sampled.

When the consumed amount of ethylene reached 2.00 kg, a small amount of the polymerization solution was sampled. When the consumed amount of ethylene reached 2.50 kg, the supplementation of the ethylene was temporarily stopped, and after the ethylene was consumed until the pressure reached 0.565 MPaG, polymerization was performed while maintaining the pressure at 0.565 MPaG. When the consumed amount of ethylene reached 3.00 kg, a small amount of the polymerization solution was sampled.

The supplementation of the ethylene was temporarily stopped, and after the ethylene was consumed until the pressure reached 0.500 MPaG, polymerization was performed while maintaining the pressure at 0.500 MPaG. When the consumed amount of ethylene reached 3.5 kg, the supplementation of the ethylene was temporarily stopped, and after the ethylene was consumed until the pressure reached 0.415 MPaG, polymerization was performed while maintaining the pressure at 0.415 MPaG. When the consumed amount of ethylene reached 4.00 kg, a small amount of the polymerization solution was sampled.

The supplementation of the ethylene was temporarily stopped, and after the ethylene was consumed until the pressure reached 0.365 MPaG, polymerization was performed while maintaining the pressure at 0.365 MPaG. When the consumed amount of ethylene reached 4.30 kg, the supplementation of the ethylene was temporarily stopped, and after the ethylene was consumed until the pressure reached 0.300 MPaG, polymerization was performed while maintaining the pressure at 0.300 MPaG. When the consumed amount of ethylene reached 4.50 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C., 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (III) in pellet form. The resulting cross-copolymer (III) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weights of the cross chains. The analysis results thereof are shown in Table 1. Additionally, the differential scanning calorimetry (DSC) results are shown in Table 4.

### Synthesis Example 4

### Synthesis of Cross-Copolymer (IV)

### (Coordination Polymerization Step)

Polymerization was performed using an autoclave with a capacity of 50 L. equipped with a stirrer and a heating/cooling jacket. 20.5 kg of cyclohexane, 2.85 kg of styrene, and divinylbenzene manufactured by Nippon Steel Chemical (meta- and para-mixture, 112 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was introduced and the pressure reached 0.455 MPaG. Thereafter, 50 mL of a toluene solution into which were dissolved 110 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.455 MPaG. When the consumed amount of ethylene reached 1.00 kg, a small amount of the polymerization solution was sampled. When the consumed amount of ethylene reached 2.00 kg, a small amount of the polymerization solution was sampled.

When the consumed amount of ethylene reached 2.10 kg, 0.19 kg of styrene was added. When the consumed amount of ethylene reached 2.70 kg, the supplementation of the ethylene was temporarily stopped, and after the ethylene was consumed until the pressure reached 0.365 MPaG, polymerization was performed while maintaining the pressure at 0.365 MPaG. When the consumed amount of ethylene reached 3.00 kg, a small amount of the polymerization solution was sampled.

When the consumed amount of ethylene reached 3.20 kg, the supplementation of the ethylene was temporarily stopped, and after the ethylene was consumed until the pressure reached 0.315 MPaG, polymerization was performed while maintaining the pressure at 0.315 MPaG. When the consumed amount of ethylene reached 3.50 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C., 0.1 kg of styrene was added, after which 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (IV) in pellet form. The resulting cross-copolymer (IV) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weight of the cross-chains. The analysis results thereof are shown in Table 1. Additionally, the differential scanning calorimetry (DSC) results are shown in Table 4.

### Synthesis Example 5

### Synthesis of Cross-Copolymer (V)

### (Coordination Polymerization Step)

Polymerization was performed using an autoclave with a capacity of 50 L, equipped with a stirrer and a heating/cooling jacket. 19.4 kg of cyclohexane, 4.79 kg of styrene, and divinylbenzene manufactured by Nippon Steel Chemical (meta- and para-mixture, 71 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was introduced and the pressure reached 0.425 MPaG. Thereafter, 50 mL of a toluene solution into which were dissolved 110 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.425 MPaG. When the consumed amount of ethylene reached 1.00 kg, a small amount of the polymerization solution was sampled. When the consumed amount of ethylene reached 2.00 kg, a small amount of the polymerization solution was sampled. When the consumed amount of ethylene reached 3.00 kg, a small amount of the polymerization solution was sampled.

When the consumed amount of ethylene reached 3.10 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C., 0.8 kg of styrene was added, after which 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (V) in pellet form. The resulting cross-copolymer (V) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weight of the cross-chains. The analysis results thereof are shown in Table 1. Additionally, the differential scanning calorimetry (DSC) results are shown in Table 4.

### Synthesis Example 6

### Synthesis of Cross-Copolymer (VI)

### (Coordination Polymerization Step)

Polymerization was performed using an autoclave with a capacity of 50 L, equipped with a stirrer and a heating/cooling jacket. 20.0 kg of cyclohexane, 2.25 kg of styrene, and divinylbenzene manufactured by Nippon Steel Chemical (meta- and para-mixture, 87 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was introduced and the pressure reached 0.540 MPaG. Thereafter, 50 mL of a toluene solution into which were dissolved 95 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.540 MPaG. When the consumed amount of ethylene reached 1.00 kg, a small amount of the polymerization solution was sampled. When the consumed amount of ethylene reached 2.00 kg, a small amount of the polymerization solution was sampled. When the consumed amount of ethylene reached 3.00 kg, a small amount of the polymerization solution was sampled. When the consumed amount of ethylene reached 4.00 kg, a small amount of the polymerization solution was sampled.

When the consumed amount of ethylene reached 4.40 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C., 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (VI) in pellet form. The resulting cross-copolymer (VI) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weight of the cross-chains. The analysis results thereof are shown in Table 1. Additionally, the differential scanning calorimetry (DSC) results are shown in Table 4.

### Synthesis Example 7

### Synthesis of Cross-Copolymer (VII)

### (Coordination Polymerization Step)

Polymerization was performed using an autoclave with a capacity of 50 L. equipped with a stirrer and a heating/cooling jacket. 20.0 kg of cyclohexane, 2.51 kg of styrene, and divinylbenzene manufactured by Nippon Steel Chemical (meta- and para-mixture, 112 mmol as divinylbenzene) were loaded and stirred at 220 rpm with the internal temperature at 60° C. Next, 50 mmol of triisobutylaluminum and 65 mmol of methylalumoxane (manufactured by Toso FineChem, MMAO-3A/toluene solution) in terms of Al were added, and the gas in the system was immediately substituted with ethylene. After the substitution, the internal temperature was raised to 90° C. and ethylene was introduced and the pressure reached 0.390 MPaG. Thereafter, 50 mL of a toluene solution into which were dissolved 110 μmol of rac-dimethylmethylene bis(4,5-benzo-1-indenyl)zirconium dichloride and 1 mmol of triisobutylaluminum was added to the autoclave. Polymerization immediately began, and the internal temperature rose to 95° C. The polymerization was carried out with the internal temperature at 95° C. and by supplementing ethylene to maintain a pressure of 0.390 MPaG. When the consumed amount of ethylene reached 1.00 kg, a small amount of the polymerization solution was sampled. When the consumed amount of ethylene reached 2.00 kg, a small amount of the polymerization solution was sampled.

When the consumed amount of ethylene reached 2.90 kg, a small amount (50 mL) of the polymerization solution was sampled, the supply of ethylene to the polymerization tank was stopped, and the ethylene was released from pressure while also cooling the internal temperature to 70° C., thereby stopping the coordination polymerization. The resin rate, the respective monomer unit contents (mol %), and the weight-average molecular weight of the aromatic vinyl/olefin-based copolymers were determined by analysis in the same manner as in Synthesis Example 1. The analysis results are shown in Tables 1 and 2.

### (Anionic Polymerization Step)

After coordination polymerization, when the internal temperature fell to 70° C., 240 mmol of n-butyllithium (hexane solution) was fed from a catalyst tank into the polymerization tank together with nitrogen gas. Anionic polymerization began immediately and the internal temperature rose from 70° C. to 75° C. at one point. Thereafter, the internal temperature was maintained at 75° C. for one hour, and the anionic polymerization was completed. After the polymerization ended, the n-butyllithium was deactivated by introducing approximately 100 mL of water.

### (Cross-Copolymer Recovery Step)

After the anionic polymerization, the polymerization solution was continuously fed into a twin-screw devolatilizing extruder using a gear pump, the solvent and deactivating water were removed by devolatilization, and the resulting material was extruded in strands and cut to obtain the cross-copolymer (VII) in pellet form. The resulting cross-copolymer (VII) was analyzed in the same manner as in Synthesis Example 1 to determine the amount (mass %) of the aromatic vinyl/olefin-based copolymers, forming the main chains, contained therein, the amount (mass %) of polymers comprising aromatic vinyl monomer units, forming the cross-chains, contained therein, and the weight-average molecular weight of the cross-chains. The analysis results thereof are shown in Table 1. Additionally, the differential scanning calorimetry (DSC) results are shown in Table 4.

### [Analysis]

### (Aromatic Vinyl/Olefin-Based Copolymer Resin Rate Measurement)

Six g of each sampled polymerization solution were mixed with 500 mL of methanol to cause precipitation of the resin, after which the precipitated resin was filtered and the resulting resin was dried. The mass of the dried resin was used to determine the resin rate:

[(Mass of dried resin)/(Mass of polymerization solution sample)]×100%

The mass of the aromatic vinyl/olefin-based copolymer that was produced by the time of sampling was determined from the analysis value obtained by measurement and the polymerization solution amount. The results are shown in Tables 1 and 2.

### (Measurement of Monomer Unit Content in Main Chains)

The styrene monomer unit content (mol %), ethylene monomer unit content (mol %), and divinylbenzene monomer unit content (mol %) in the aromatic vinyl/olefin-based copolymers in the coordination polymerization step, obtained for each of Synthesis Examples 1-7, were measured by the following method.
Equipment: Avance 300 (manufactured by Bruker)
Procedure: A resin sample precipitated in methanol was dissolved in heavy 1,1,2,2-tetrachloroethane and measured by ¹H-NMR at 130° C. The styrene monomer unit content (mol %) was determined from a comparison of the area-based intensity of peaks due to phenyl group protons using trimethylsilane as the standard. The ethylene monomer unit content (mol %) was determined from a comparison of the area-based intensity of peaks due to olefin protons. The divinylbenzene monomer unit content (mol %) was determined from a comparison of the area-based intensity of peaks due to vinyl group protons. The results are shown in Tables 1 and 2.

### (Weight-Average Molecular Weight of Main Chains)

The weight-average molecular weights of the aromatic vinyl/olefin-based copolymers obtained in the coordination polymerization step in Synthesis Examples 1-7 are polystyrene-converted values measured by gel permeation chromatography (GPC), the measurements being made under the below-indicated measurement conditions. The results are shown in Table 1.
Equipment: HLC-8220 (manufactured by Toso)
Column: Shodex GPC KF-404HQ, four in a serial arrangement
Temperature: 40° C.
Detection: Differential refractive index
Solvent: Tetrahydrofuran
Calibration curve: Produced using standard polystyrene (PS)

### (Main Chain and Cross-Chain Content)

In the cross-copolymers (I) to (VII) obtained in Synthesis Examples 1 to 7, the amounts (mass %) of the aromatic vinyl/olefin-based copolymers; which are the main chains, and the amounts (mass %) of the polymers comprising the aromatic vinyl monomer units, which are the cross-chains, were determined in the same manner as the composition analysis of the aromatic vinyl/olefin-based copolymers, by determining the styrene monomer unit content and the ethylene monomer unit content by ¹H-NMR, and using the previously determined aromatic vinyl/olefin-based copolymer main chain compositions to calculate the main chain content (mass %) and the cross-chain content (mass %). The results are shown in Table 1.

### (Weight-Average Molecular Weight of Cross-Chains)

In the cross-copolymers (I) to (VII) obtained in Synthesis Example 1 to 7, the polymers comprising aromatic vinyl monomer units that are not bound to the main chains, which are produced in small amounts as byproducts of the anionic polymerization step, were separated and extracted by the method described below.
(i) The pellets were dissolved in toluene;
(ii) the toluene solution in (i) was dripped into acetone while stirring;
(iii) the acetone solution in (ii) was filtered and separated into soluble components and insoluble components;
(iv) the insoluble component solution in (iii) was dripped into methanol while stirring; and
(v) the precipitate in the methanol solution in (iv) was filtered, isolated, and vacuum-dried to obtain a polymer powder comprising aromatic vinyl monomer units.

Due to the nature of anionic polymerization, the polymers comprising aromatic vinyl monomer units forming the cross-chains and the polymers comprising aromatic vinyl monomer units not bonding to the main chains have approximately the same molecular weight. Therefore, the weight-average molecular weights of the cross-chains were determined by measuring the weight-average molecular weights of the isolated polymers comprising the isolated aromatic monomer units. The weight-average molecular weights are polystyrene-converted values measured by gel permeation chromatography (GPC), the measurements being made under the below-indicated measurement conditions. The results are shown in Table 1.
Equipment: HLC-8220 (manufactured by Toso)
Column: Shodex GPC KF-404HQ, four in a serial arrangement
Temperature: 40° C.
Detection: Differential refractive index
Solvent: Tetrahydrofuran
Calibration curve: Produced using standard polystyrene (PS)

TABLE 1

| | | Aromatic Vinyl/Olefin-based Copolymer Analysis Results | | | | | | Cross-copolymer | | |
|---|---|---|---|---|---|---|---|---|---|---|
| | | | | Divinyl | | | | Analysis Results | | |
| | | Styrene | Ethylene | benzene | Main | | | | | Cross- |
| | | monomer | monomer | monomer | chain | | | Main | Cross- | chain |
| | | unit | unit | unit | wt.-avg. | | Resin | chain | chain | wt.-avg. |
| | | content | content | content | mol. wt. | Resin rate | mass | content | content | mol. wt. |
| | | [mol %] | [mol %] | [mol %] | [g/mol] | [wt %] | [kg] | [mass %] | [mass %] | [g/mol] |
| Synthesis | Cross-copolymer | 10.6 | 89.3 | 0.06 | 109,000 | 20.7 | 5.42 | 87.7 | 12.3 | 24,000 |
| Example 1 | (I) | | | | | | | | | |
| Synthesis | Cross-copolymer | 12.5 | 87.4 | 0.06 | 118,000 | 19.6 | 5.12 | 87.6 | 12.4 | 23,000 |
| Example 2 | (II) | | | | | | | | | |
| Synthesis | Cross-copolymer | 9.2 | 90.8 | 0.05 | 97,000 | 22.9 | 6.15 | 91.5 | 8.5 | 24,000 |
| Example 3 | (III) | | | | | | | | | |
| Synthesis | Cross-copolymer | 15.0 | 84.9 | 0.08 | 121,000 | 21.2 | 5.74 | 88.0 | 12.0 | 22,000 |
| Example 4 | (IV) | | | | | | | | | |
| Synthesis | Cross-copolymer | 24.0 | 76.0 | 0.04 | 115,000 | 24.4 | 6.66 | 77.0 | 23.0 | 26,000 |
| Example 5 | (V) | | | | | | | | | |
| Synthesis | Cross-copolymer | 9.2 | 90.8 | 0.05 | 90,000 | 22.7 | 6.05 | 91.5 | 8.5 | 24,000 |
| Example 6 | (VI) | | | | | | | | | |
| Synthesis | Cross-copolymer | 15.0 | 84.9 | 0.08 | 119,000 | 18.6 | 4.73 | 88.0 | 12.0 | 22,000 |
| Example 7 | (VII) | | | | | | | | | |

TABLE 2

| | | Aromatic Vinyl/Olefin-based Copolymer | | | | | Aromatic Vinyl/Olefin-based Copolymer | | | | |
|---|---|---|---|---|---|---|---|---|---|---|---|
| | | Analysis Results at 1.00 kg Ethylene Consumed | | | | | Analysis Results at 2.00 kg Ethylene Consumed | | | | |
| | | | | Divinyl | | | | | Divinyl | | |
| | | Styrene | Ethylene | benzene | | | Styrene | Ethylene | benzene | | |
| | | monomer | monomer | monomer | | | monomer | monomer | monomer | | |
| | | unit | unit | unit | Resin | Resin | unit | unit | unit | Resin | Resin |
| | | content | content | content | rate | mass | content | content | content | rate | mass |
| | | [mol %] | [mol %] | [mol %] | [wt %] | [kg] | [mol %] | [mol %] | [mol %] | [wt %] | [kg] |
| Synthesis | Cross- | 12.6 | 87.3 | 0.07 | 6.3 | 1.47 | 11.4 | 88.6 | 0.06 | 11.9 | 2.90 |
| Example 1 | copolymer | | | | | | | | | | |
| | (I) | | | | | | | | | | |
| Synthesis | Cross- | 12.8 | 87.1 | 0.08 | 6.3 | 1.50 | 12.6 | 87.3 | 0.07 | 12.2 | 3.02 |
| Example 2 | copolymer | | | | | | | | | | |
| | (II) | | | | | | | | | | |
| Synthesis | Cross- | 11.8 | 88.1 | 0.08 | 6.3 | 1.46 | 10.6 | 89.3 | 0.06 | 11.7 | 2.84 |
| Example 3 | copolymer | | | | | | | | | | |
| | (III) | | | | | | | | | | |
| Synthesis | Cross- | 20 | 79.9 | 0.12 | 7.8 | 1.85 | 17.3 | 82.7 | 0.10 | 13.7 | 3.48 |
| Example 4 | copolymer | | | | | | | | | | |
| | (IV) | | | | | | | | | | |
| Synthesis | Cross- | 32 | 67.9 | 0.07 | 10.6 | 2.66 | 27.8 | 72.1 | 0.06 | 18.3 | 4.79 |
| Example 5 | copolymer | | | | | | | | | | |
| | (V) | | | | | | | | | | |
| Synthesis | Cross- | 14.3 | 85.6 | 0.09 | 6.8 | 1.57 | 12.6 | 87.4 | 0.07 | 12.5 | 3.03 |
| Example 6 | copolymer | | | | | | | | | | |
| | (VI) | | | | | | | | | | |
| Synthesis | Cross- | 20.8 | 79.1 | 0.11 | 8.0 | 1.89 | 17.4 | 82.5 | 0.08 | 14.2 | 3.49 |
| Example 7 | copolymer | | | | | | | | | | |
| | (VII) | | | | | | | | | | |
| | | Aromatic Vinyl/Olefin-based Copolymer | | | | | Aromatic Vinyl/Olefin-based Copolymer | | | | |
| | | Analysis Results at 3.00 kg Ethylene Consumed | | | | | Analysis Results at 4.00 kg Ethylene Consumed | | | | |
| | | | | Divinyl | | | | | Divinyl | | |
| | | Styrene | Ethylene | benzene | | | Styrene | Ethylene | benzene | | |
| | | monomer | monomer | monomer | | | monomer | monomer | monomer | | |
| | | unit | unit | unit | Resin | Resin | unit | unit | unit | Resin | Resin |
| | | content | content | content | rate | mass | content | content | content | rate | mass |
| | | [mol %] | [mol %] | [mol %] | [wt %] | [kg] | [mol %] | [mol %] | [mol %] | [wt %] | [kg] |
| | Synthesis | 10.8 | 89.1 | 0.05 | 16.9 | 4.30 | — | — | — | — | — |
| | Example 1 | | | | | | | | | | |
| | Synthesis | 12.4 | 87.5 | 0.06 | 17.6 | 4.53 | — | — | — | — | — |
| | Example 2 | | | | | | | | | | |
| | Synthesis | 9.8 | 90.2 | 0.05 | 16.5 | 4.16 | 9.4 | 90.6 | 0.05 | 20.9 | 5.49 |
| | Example 3 | | | | | | | | | | |
| | Synthesis | 15.6 | 84.3 | 0.08 | 18.8 | 5.00 | — | — | — | — | — |
| | Example 4 | | | | | | | | | | |
| | Synthesis | 24.3 | 75.7 | 0.05 | 23.8 | 6.48 | — | — | — | — | — |
| | Example 5 | | | | | | | | | | |
| | Synthesis | 11.1 | 88.8 | 0.06 | 17.2 | 4.35 | 9.9 | 90.1 | 0.05 | 21.3 | 5.58 |
| | Example 6 | | | | | | | | | | |
| | Synthesis | — | — | — | — | — | — | — | — | — | — |
| | Example 7 | | | | | | | | | | |

### (Calculation of Ethylene Monomer Unit Content (Mol %) in Main Chains During Coordination Polymerization)

The values indicated in (a) to (e) below were calculated by means of methods (1) to (3) below:
(a) the ethylene monomer unit content (mol %) in the aromatic vinyl/olefin-based copolymer obtained at the time that 1.00 kg of ethylene was consumed, and the mass % thereof relative to the aromatic vinyl/olefin-based copolymer obtained at the time the coordination polymerization of the copolymer stopped;
(b) the ethylene monomer unit content (mol %) in the aromatic vinyl/olefin-based copolymer obtained at the time that 1.01 kg to 2.00 kg of ethylene was consumed, and the mass % thereof with respect to the aromatic vinyl/olefin-based copolymer obtained at the time the coordination polymerization of the copolymer stopped;
(c) the ethylene monomer unit content (mol %) in the aromatic vinyl/olefin-based copolymer obtained at the time that 2.01 kg to 3.00 kg of ethylene was consumed, or at the time the coordination polymerization stopped, and the mass % thereof with respect to the aromatic vinyl/olefin-based copolymer obtained at the time the coordination polymerization of the copolymer stopped;
(d) the ethylene monomer unit content (mol %) in the aromatic vinyl/olefin-based copolymer obtained at the time that 3.01 kg to 4.00 kg of ethylene was consumed, or at the time the coordination polymerization stopped, and the mass % thereof with respect to the aromatic vinyl/olefin-based copolymer obtained at the time the coordination polymerization of the copolymer stopped;
(e) the ethylene monomer unit content (mol %) in the aromatic vinyl/olefin-based copolymer obtained at the time that 4.01 kg of ethylene was consumed, or at the time the coordination polymerization stopped, and the mass % thereof with respect to the aromatic vinyl/olefin-based copolymer obtained at the time the coordination polymerization of the copolymer stopped.

The above-mentioned values (a) to (e) were calculated by the following methods (1) to (3).
(1) The respective monomer unit contents (mol %) in the aromatic vinyl/olefin-based copolymers produced at the time of sampling, and the amount (kg) of the respective monomer units in the copolymers, based on the mass (kg) of the copolymers, were determined.
(2) The amounts (kg) of the respective monomer units in the aromatic vinyl/olefin-based copolymers were determined for (a), (b), (c), (d) and (e).
(a): The values determined in (1) at the time 1.00 kg of ethylene (Et) was consumed are used.
(b): (The values determined in (1) at the time 2.00 kg of ethylene was consumed)—(the values determined in (1) at the time 1.00 kg of ethylene was consumed)
(c): (The values determined in (1) at the time 3.00 kg of ethylene was consumed, or at the time the coordination polymerization stopped)—(the values determined in (1) at the time 2.00 kg of ethylene was consumed)
(d): (The values determined in (1) at the time 4.00 kg of ethylene was consumed, or at the time the coordination polymerization stopped)—(the values determined in (1) at the time 3.00 kg of ethylene was consumed)
(e): (The values determined in (1) at the time the coordination polymerization stopped)—(the values determined in (1) at the time 4.00 kg of ethylene was consumed)
(3) The ethylene monomer unit contents (mol %) for (a), (b), (c), (d) and (e) were determined from the amounts (kg) of the respective monomer units determined in (2). The results are shown in Table 3.
(4) The sum of the amounts (kg) of the monomer units determined in (2) is divided by the mass of the aromatic vinyl/olefin-based copolymers obtained at the time the coordination polymerization stopped, thereby determining the mass % of the aromatic vinyl/olefin-based copolymers for (a), (b), (c), (d) and (e) relative to the aromatic vinyl/olefin-based copolymers obtained at the time coordination polymerization stopped. The results are shown in Table 3.

TABLE 3

| | | | | | | Copolymer Analysis Results | |
|---|---|---|---|---|---|---|---|
| | | | | Copolymer Analysis Results | | When 2.01 to 3.00 kg Et | |
| | | Copolymer Analysis Results | | When 1.01 to 2.00 kg Et | | Consumed or Coordination | |
| | | When 1.00 kg Et Consumed | | Consumed | | Polymerization Stopped | |
| | | | Mass % relative | | Mass % relative | | Mass % relative |
| | | Ethylene | to copolymer | Ethylene | to copolymer | Ethylene | to copolymer |
| | | monomer | when | monomer | when | monomer | when |
| | | unit | coordination | unit | coordination | unit | coordination |
| | | content | polymerization | content | polymerization | content | polymerization |
| | | [mol %] | stopped | [mol %] | stopped | [mol %] | stopped |
| Synthesis | Cross-copolymer | 87.3 | 27.1 | 89.7 | 26.4 | 90.3 | 25.8 |
| Example 1 | (I) | | | | | | |
| Synthesis | Cross-copolymer | 87.1 | 29.3 | 87.5 | 29.7 | 87.9 | 29.5 |
| Example 2 | (II) | | | | | | |
| Synthesis | Cross-copolymer | 88.1 | 23.7 | 90.6 | 22.4 | 91.8 | 21.5 |
| Example 3 | (III) | | | | | | |
| Synthesis | Cross-copolymer | 79.9 | 32.2 | 85.4 | 28.4 | 87.8 | 26.5 |
| Example 4 | (IV) | | | | | | |
| Synthesis | Cross-copolymer | 67.9 | 39.9 | 76.7 | 32.0 | 83.9 | 25.4 |
| Example 5 | (V) | | | | | | |
| Synthesis | Cross-copolymer | 85.6 | 26.0 | 89.1 | 24.1 | 92.0 | 21.8 |
| Example 6 | (VI) | | | | | | |
| Synthesis | Cross-copolymer | 79.1 | 40.0 | 86.1 | 33.8 | 90.7 | 26.2 |
| Example 7 | (VII) | | | | | | |

| | Copolymer Analysis Results | | Copolymer Analysis Results | |
|---|---|---|---|---|
| | When 3.01 to 4.00 kg Et | | From 4.01 kg Et Consumed | |
| | Consumed or Coordination | | to When Coordination | |
| | Polymerization Stopped | | Polymerization Stopped | |
| | | Mass % relative | | Mass % relative |
| | Ethylene | to copolymer | Ethylene | to copolymer |
| | monomer | when | monomer | when |
| | unit | coordination | unit | coordination |
| | content | polymerization | content | polymerization |
| | [mol %] | stopped | [mol %] | stopped |
| Synthesis | 90.1 | 20.7 | — | — |
| Example 1 | | | | |
| Synthesis | 87.7 | 11.5 | — | — |
| Example 2 | | | | |
| Synthesis | 91.8 | 21.6 | 92.3 | 10.8 |
| Example 3 | | | | |
| Synthesis | 88.1 | 12.9 | — | — |
| Example 4 | | | | |
| Synthesis | 87.7 | 2.7 | — | — |
| Example 5 | | | | |
| Synthesis | 93.9 | 20.3 | 95.7 | 7.8 |
| Example 6 | | | | |
| Synthesis | — | — | — | — |
| Example 7 | | | | |

### (Tm (Melting Peak Temperature))

Tm (the melting peak temperature) was measured based on differential scanning calorimetry (DSC) that involved cooling to −50° C. in a 30 mL/min nitrogen flow, then heating to 180° C. at a temperature increase rate of 10° C./min, re-cooling to −50° C., and heating to 180° C. at a temperature increase rate of 10° C./min. The results are shown in Table 4.

### (Heat of Fusion)

The heat of fusion was measured by calculation from the area of the DSC curve between −20° C. and 130° C. using a straight line drawn between the points on the DSC curve at −20° C. and 130° C. The results are shown in Table 4.

TABLE 4

| | | | Heat of |
|---|---|---|---|
| | | Tm | Fusion |
| | | ° C. | J/g |
| Synthesis Example 1 | Cross-copolymer (I) | 71 | 68 |
| Synthesis Example 2 | Cross-copolymer (II) | 67 | 54 |
| Synthesis Example 3 | Cross-copolymer (III) | 77 | 72 |
| Synthesis Example 4 | Cross-copolymer (IV) | 63 | 48 |
| Synthesis Example 5 | Cross-copolymer (V) | 18 | 3 |
| Synthesis Example 6 | Cross-copolymer (VI) | 88 | 74 |
| Synthesis Example 7 | Cross-copolymer (VII) | 55 | 40 |

### Example 1 to Example 4, Comparative Examples 1-3

Test pieces in accordance with the following evaluation criteria were prepared for the cross-copolymers obtained in Synthesis Examples 1 to 7, and evaluated. The results are shown in Table 5.

### (Hardness)

An instantaneous value for the hardness was determined by using Type A durometer hardness in compliance with the JIS K6253 standard. A Type A hardness of 85 or lower was evaluated as being of passing level.

### (Tensile Properties)

The 50% modulus and the breaking strength were determined in compliance with the JIS K6251 standard. The 50% modulus refers to the tensile stress when an elongation of 50% is applied to the test piece. As the test piece, a 1 mm-thick pressed sheet was punched out in the shape of a Type 3 dumbbell, and used. The pulling speed was 500 mm/min. A 50% modulus of 3.5 MPa or higher and a breaking strength of 30 MPa or higher were evaluated as being of passing level.

### (Total Light Transmittance)

A square mirror-surface pressed sheet that was 1 mm thick and 50 mm on a side was measured using a haze meter (Nippon Denshoku Industries NDH-1001DP) in compliance with the JIS K7136 standard. A value of 82% or higher was evaluated as being of passing level.

### (Anti-Blocking Properties)

Four square mirror-surface pressed sheets, each 1 mm thick and 60 mm on a side, were stacked, a disc-shaped 100 g weight having a diameter of 60 min was placed thereon, and the peelability of the four pressed sheets after 24 hours at 23° C. was evaluated according to the following criteria:
3: The four pressed sheets did not stick together and could be cleanly peeled away.
2: Two or three of the pressed sheets stuck together and were difficult to peel away, but one or two of the pressed sheets did not stick together and could be cleanly peeled away.
1: The four pressed sheets stuck together and were difficult to peel away.

TABLE 5

| | | | | | | | Comparative | Comparative | Comparative |
|---|---|---|---|---|---|---|---|---|---|
| | | | Example 1 | Example 2 | Example 3 | Example 4 | Example 1 | Example 2 | Example 3 |
| | | | Cross- | Cross- | Cross- | Cross- | Cross- | Cross- | Cross- |
| | | | copolymer | copolymer | copolymer | copolymer | copolymer | copolymer | copolymer |
| | | | (I) | (II) | (III) | (IV) | (V) | (VI) | (VII) |
| Type A Hardness | | — | 81 | 79 | 83 | 78 | 58 | 90 | 80 |
| Tensile | 50% | MPa | 4.9 | 4.3 | 5.3 | 3.8 | 1.9 | 5.4 | 3.3 |
| Properties | Modulus | | | | | | | | |
| | Breaking | MPa | 39 | 37 | 41 | 35 | 17 | 42 | 20 |
| | Strength | | | | | | | | |

| | | | | | | | | |
|---|---|---|---|---|---|---|---|---|
| Anti-blocking | — | 3 | 3 | 3 | 2 | 1 | 2 | 3 |
| Properties | | | | | | | | |
| Total Light | % | 85.8 | 86.8 | 83.8 | 85.9 | 84.2 | 80.2 | 84.7 |
| Transmittance | | | | | | | | |

For the cross-polymers of Examples 1 to 4, all four had a Type A hardness of 85 or lower, a 50% modulus of 3.5 MPa or higher, a tensile breaking strength of 30 MPa or higher and a total light transmittance of 82% or higher, thus exhibiting excellent softness, tensile properties, transparency. and anti-blocking properties. On the other hand, Comparative Examples 1 to 3 were inferior in one of the physical properties of softness, tensile properties, transparency, and anti-blocking properties.

### [Evaluation of Single-Layer Tube]

The cross-copolymers obtained in Examples 1 to 4 and Comparative Examples 1 to 3 were used to prepare, by extrusion molding, single-layer tubes having an outer diameter of 3.6 mm, an inner diameter of 2.4 mm, and a tube thickness of 0.6 mm. Each tube was evaluated for the properties as a tube, in accordance with the following criteria. The results are shown in Table 6.

### (Tube Transparency)

Biological saline solution was made to flow through the tubes and the tubes were observed as to whether the liquid surfaces, bubbles and the like could be perceived by the naked eye. A grade of 3 was recorded if they could be easily observed, a grade of 2 was recorded if they could be observed but were slightly difficult to perceive, and a grade of 1 was recorded if observation was difficult. A grade of 3 was considered to be passing.

### (Tube Blocking Properties)

Two 10 cm-long tubes were placed parallel to each other so as to overlap by 5 cm, tied together with paper tape, and subjected to high-pressure steam sterilization (121° C., 30 minutes). Thereafter, the tied paper tape was removed and the shear peel strength between the tubes was measured. The shear peel strength was measured using a tensile testing machine at a test speed of 100 mm/min. A value of 5 N or lower was considered to be passing. If the two tubes did not stick and came apart when the tied paper tape was removed, the value was considered to be 0 N.

### (Kink Start Radius)

A 20 cm tube was bent to various radii of curvature, and the radius of curvature at which a kink was formed in the tube after one minute was determined. A value of 10 mm or less was considered to be passing.

### (Forceps Resistance)

At 40° C., a tube filled with biological saline solution was occluded for 15 hours using medical tube forceps, after which the forceps were removed and the time until the shape of the inside of the tube recovered and the tube became passable was measured, and evaluated according to the following criteria.
4: The shape recovered and the tube became passable within three seconds.
3: The shape recovered and the tube became passable in 3-10 seconds.
2: At least 10 seconds elapsed before the shape recovered and the tube became passable.
1: The shape did not recover and the tube did not become passable.
A grade of 4 or 3 was considered to be passing.

TABLE 6

| | | | | | Comparative | Comparative | Comparative |
|---|---|---|---|---|---|---|---|
| | Example 1 | Example 2 | Example 3 | Example 4 | Example 1 | Example 2 | Example 3 |
| | Cross- | Cross- | Cross- | Cross- | Cross- | Cross- | Cross- |
| | copolymer | copolymer | copolymer | copolymer | copolymer | copolymer | copolymer |
| | (I) | (II) | (III) | (IV) | (V) | (VI) | (VII) |
| Tube | 3 | 3 | 3 | 3 | 3 | 2 | 3 |
| Transparency | | | | | | | |
| Tube Blocking | 0 N: pass | 0 N: pass | 0 N: pass | 3 N: pass | 47 N: fail | 0 N: pass | 30 N: fail |
| Properties | | | | | | | |
| Kink Start | 8 mm: pass | 8 mm: pass | 8 mm: pass | 8 mm: pass | 9 mm: pass | 13 mm: fail | 8 mm: pass |
| Radius | | | | | | | |
| Forceps | 4 | 4 | 4 | 3 | 1 | 4 | 2 |
| Resistance | | | | | | | |

The single-layer tubes using the cross-copolymers of Examples 1 to 4 exhibited good transparency, anti-blocking properties, good kink properties, and forceps resistance, and thus exhibited excellent properties for use as medical single-layer tubes. On the other hand, the single-layer tubes using the cross-copolymers of Comparative Examples 1 to 3 were inferior in one of the physical properties of transparency, anti-blocking properties, kink properties, and forceps resistance.

### INDUSTRIAL APPLICABILITY

According to the present invention, it is possible to provide a medical single-layer tube having excellent transparency, anti-blocking properties, kink properties, and forceps resistance.

1. A cross-copolymer containing:
by 75-95 mass %, main chains comprising aromatic vinyl/olefin-based copolymers comprising 8.99-15.99 mol % of aromatic vinyl monomer units, 84-91 mol % of olefin monomer units and 0.01-0.5 mol % of aromatic polyene monomer units; and by 5-25 mass %, cross-chains comprising polymers comprising aromatic vinyl monomer units; wherein,
based on differential scanning calorimetry (DSC) in which the cross-copolymer is cooled to −50° C. in a 30 mL/min nitrogen flow, then heated to 180° C. at a temperature increase rate of 10° C./min, re-cooled to −50° C., and heated to 180° C. at a temperature increase rate of 10° C./min, the cross-copolymer has Tm (a top temperature of melting peak) of 60-80° C., and a heat of fusion of 45-75 J/g, as calculated from the area of the DSC curve between −20° C. and 130° C. by using a straight line drawn between points on the DSC curve at −20° C. and 130° C.

2. The cross-copolymer of claim 1, wherein the composition distribution of the aromatic vinyl/olefin-based copolymers forming the main chains is such that aromatic vinyl/olefin-based copolymers comprising at least 85 mol % and at most 92 mol % of olefin monomer units are contained in an amount of at least 50 mass %, aromatic vinyl/olefin-based copolymers comprising less than 85 mol % of olefin monomer units are contained in an amount of less than 35 mass %, and aromatic vinyl/olefin-based copolymers comprising more than 92 mol % of olefin monomer units are contained in an amount of less than 15 mass %.

3. The cross-copolymer of claim 1, wherein Tm (the top temperature of melting peak) is 65-73° C. and the heat of fusion is 50-70 J/g.

4. The cross-copolymer of claim 1, wherein the aromatic vinyl monomer units are styrene.

5. The cross-copolymer of claim 1, wherein the olefin monomer units are ethylene.

6. A medical single-layer tube containing the cross-copolymer of claim 1.
